(19) United States
(12) Patent Application Publication
(10) Pub. No.: US 2021/0386144 A1
(43) Pub. Date: Dec. 16, 2021

(54) ANTI-VIRUS FACE MASK AND FILTER

(71) Applicant: Electro-Mask, LLC, Bellevue, WA (US)

(72) Inventors: CHRISTOPHER A. WIKLOF, EVERETT, WA (US); Donald Kendrick, BELLEVUE, WA (US)

(21) Appl. No.: 17/348,461

(22) Filed: Jun. 15, 2021

Related U.S. Application Data
(60) Provisional application No. 63/039,434, filed on Jun. 15, 2020.

(51) Int. Cl. A41D 13/11; A61L 9/22

(52) U.S. Cl. CPC A41D 13/1192; A41D 13/1161; A61L 2209/21; A41D 2500/20; A41D 2400/00; A61L 9/22

(57) ABSTRACT

A face mask and face mask filter system filters droplets and aerosols, and applies a treatment that causes viruses particles carried by droplets and aerosols to be rendered non-infectious by application of a low voltage electric field. Optionally, the face mask filter system includes an electric circuit operatively coupled to the face mask filter and configured to monitor and/or enhance treatment of virus particles.

### CROSS-REFERENCE TO RELATED APPLICATIONS

The present application claims the benefit of co-pending U.S. Provisional Patent Application No. 63/039,434, entitled “ANTI-VIRUS FACE MASK AND FILTER,” filed on Jun. 15, 2020 (docket number 3074-001-02), which, to the extent not inconsistent with the disclosure herein, is incorporated by reference.

### SUMMARY

According to an embodiment, a material for use in a face mask, and a face mask formed therefrom, provides a porous substrate with appropriate filtering capability to reduce the passage therethrough of SARS-CoV-2 virus carried in an aerosol. First and second patterns of conductive materials are disposed on respective surfaces of one or more porous substrates, and the respective surfaces held in a relationship to form a low voltage electric field and/or a low current electric field between each of a plurality of electric field junctions formed between the respective first and second patterns. The presence of a low voltage electric field was found to kill or disable SARS-CoV-2 virus so as to make the remains of the virus substantially non-infectious. Accordingly, the material for use in a face mask, and a face mask formed therefrom, provides filtering to reduce the passage of droplets and aerosols carrying SARS-CoV-2 (and other viruses) and provides an electric field environment configured to render trapped virus non-infectious.

According to an embodiment, a system for filtering and treating air containing an aerosol or droplets includes a first substrate, a reducing agent formed on a first surface of the first substrate, a second porous substrate formed from a porous material, and an oxidizing agent formed on a second surface of the second porous substrate separate from the first surface. The reducing agent and the oxidizing agent are disposed in relationship to one another to form a plurality of electric field junctions between the reducing agent and the oxidizing agent. The plurality of electric field junctions collectively form a low voltage electric field across the second substrate.

According to an embodiment, a filter for inhibiting the spread of infectious disease includes at least one porous substrate, a first pattern carrying silver formed on the at least one porous substrate, and a second pattern carrying zinc formed on the at least one porous substrate. A first electrical lead may be in electrical continuity with at least a portion of the first pattern and a second electrical lead may be in electrical continuity with at least a portion of the second pattern. An electrical circuit may be operatively coupled to the first and second electrical leads. The silver and zinc may be operatively coupled to form a reduction-oxidation reaction such that at least one of electrical voltage or electrical current corresponding to the reduction-oxidation reaction between the first and second patterns is sufficient to render an infectious agent held by the porous substrate non-infectious.

### BRIEF DESCRIPTION OF THE DRAWINGS

FIG. 1 is a plan view diagram of a portion of a filter including a reducing agent on a first surface of a first substrate, according to an embodiment.

FIG. 2 is a plan view diagram of a portion of a filter including an oxidizing agent on a second surface of a second porous substrate, according to an embodiment.

FIG. 3 is a plan view diagram of a filter showing a relationship between the reducing agent and the oxidizing agent of FIGS. 1 and 2, whereby electric field junctions are formed, according to an embodiment.

FIG. 4A is a sectional view diagram of the filter of FIG. 3 showing a relationship between the reducing agent and the oxidizing agent whereby electric field junctions are formed, according to an embodiment.

FIG. 4B is a sectional view diagram of the filter of FIG. 3 showing a relationship between the reducing agent and the oxidizing agent whereby electric field junctions are formed, according to another embodiment.

FIG. 4C is a sectional view diagram of the filter of FIG. 3 showing a relationship between the reducing agent and the oxidizing agent whereby electric field junctions are formed, according to another embodiment.

FIG. 5A is a plan view of a portion of the filter of FIG. 3 showing a relationship between a conductor (e.g., reducing agent or oxidizing agent), and a dielectric on a surface of a porous substrate, according to an embodiment.

FIG. 5B is a sectional view of the portion of the filter of FIG. 5A showing a relationship between a conductor and a dielectric on a surface of a porous substrate, according to an embodiment.

FIG. 6 is a diagram of a filter system including a filter operatively coupled to an electric circuit, according to an embodiment.

FIG. 7 is a diagram of a filter system including a filter operatively coupled to an electric circuit, according to an embodiment.

FIG. 8 is a diagram of a filter system including a filter operatively coupled to an electric circuit, according to an embodiment.

FIG. 9 is a diagram of a filter system including a filter operatively coupled to an electric circuit, according to an embodiment.

FIG. 10 is a diagram of a face mask including a filter configured to filter aerosols and droplets carrying virus particles and to render the virus particles non-infectious, according to an embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description and drawings are not meant to be limiting. Other embodiments may be utilized, and other changes may be made, without departing from the spirit or scope of the subject matter presented herein.

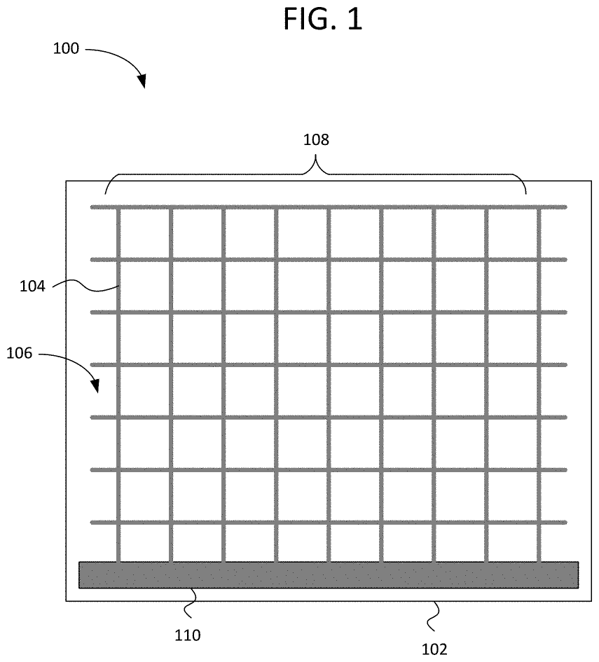

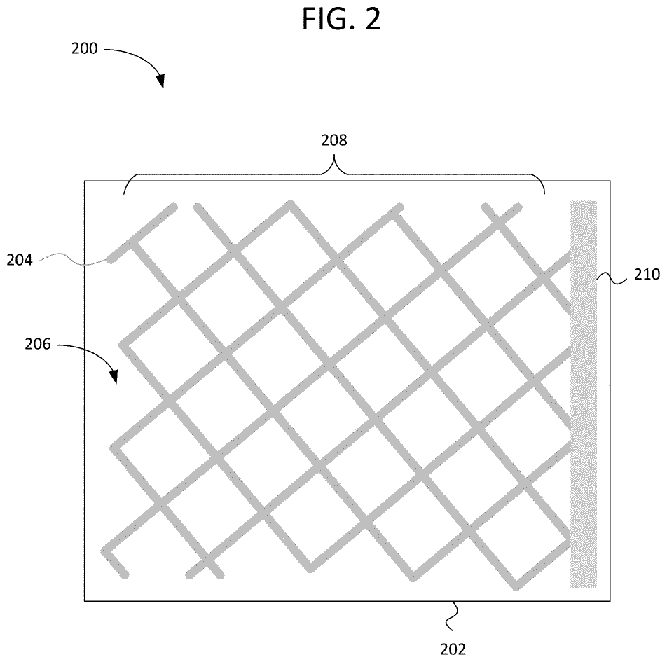

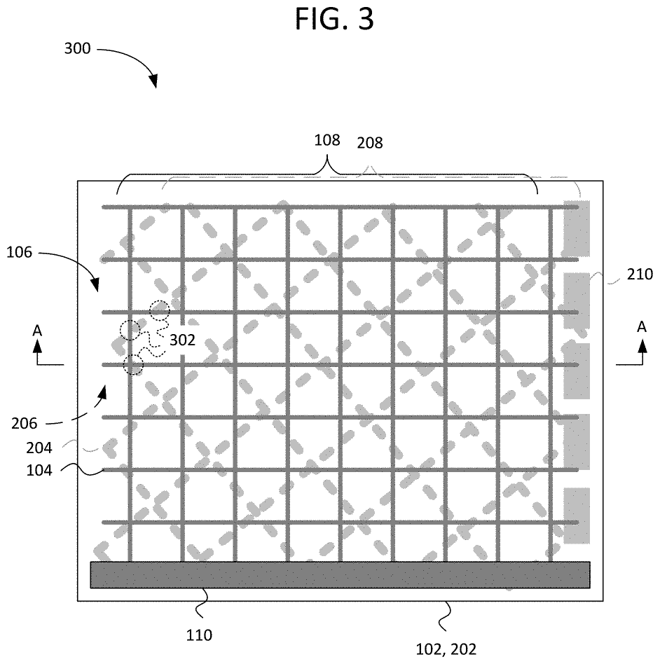

FIG. 1 is a plan view diagram of a portion of a filter 100 including a reducing agent 104 on a first surface 106 of a substrate 102, according to an embodiment. FIG. 2 is a plan view diagram of a portion of a filter 200 including an oxidizing agent 204 on a second surface 206 of a porous substrate 202, according to an embodiment. FIG. 3 is a plan view diagram of a filter 300 showing a relationship between the reducing agent 104 and the oxidizing agent 204 whereby electric field junctions 302 are formed, according to an embodiment.

According to embodiments, referring to FIGS. 1-3, a system for filtering and treating air containing an aerosol or droplets includes a first substrate 102, a reducing agent 104, such as zinc, formed on a first surface 106 of the first substrate 102, a second porous substrate 202 formed from a porous material, and an oxidizing agent 204, such as silver, formed on a second surface 206 of the second porous substrate 202 separate from the first surface 106. In one embodiment, the oxidizing agent 204 may be formed in a second pattern 208.

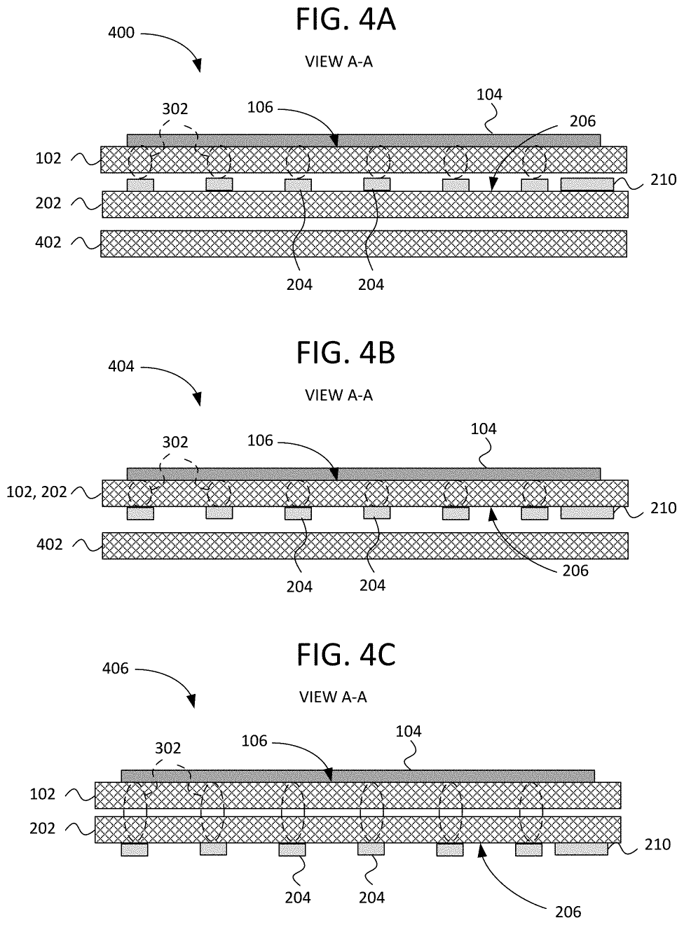

The reducing agent 104 and the oxidizing agent 204 may be disposed in relationship to one another, as shown in FIGS. 3 and 4A-4C, to form a plurality of electric field junctions 302 between the reducing agent 104 and the oxidizing agent 204. The plurality of electric field junctions 302 collectively may form a low voltage electric field across the first substrate 102.

In an embodiment, at least the second porous substrate 202 is configured to filter at least a portion of an entrained aerosol or droplet when air is passed therethrough. The low voltage electric field formed by the plurality of electrical field junctions 302 may perform treatment on an infectious particle carried by the aerosol or droplet. In one embodiment, performing the treatment includes rendering a virus carried by the aerosol or droplet non-infectious.

According to an embodiment, the reducing agent 104 includes zinc. In another embodiment, the first substrate 102 includes a wire screen. The reducing agent 104 may include a layer of zinc disposed on the first substrate 102, and the first surface 106 may include substantially the entire surface of the wire screen, not including ends of wires where the wires are cut.

According to an embodiment, the first substrate 102 includes a porous substrate. The first surface 106 of the first substrate 102 may include a first side 106 of the first substrate 102.

According to an embodiment, the reducing agent 104 includes a metal foil formed in a first pattern 108 on the first surface 106 of the first substrate 102. In another embodiment, the reducing agent 104 includes an ink carrying metal particles. Additionally and/or alternatively, the reducing agent 104 includes a paint carrying metal particles. The reducing agent 104 may be disposed in a continuous pattern 108 (i.e., in a continuous pattern having electrical continuity across the pattern 108). In one embodiment, the continuous pattern 108 includes a solid, porous patch.

According to an embodiment, the oxidizing agent 204 includes silver, Ag. In another embodiment, the oxidizing agent 204 includes silver, Ag and silver oxide Ag₂O. Additionally and/or alternatively, the oxidizing agent 204 includes an ink carrying metal particles. The oxidizing agent 204 may include a paint carrying metal particles.

FIG. 4A is a sectional view diagram of the filter 300 of FIG. 3 showing a relationship between the reducing agent 104 and the oxidizing agent 204 whereby electric field junctions 302 are formed, according to an embodiment 400. FIG. 4B is a sectional view diagram of the filter 300 of FIG. 3 showing a relationship between the reducing agent 104 and the oxidizing agent 204 whereby electric field junctions 302 are formed, according to another embodiment 404. FIG. 4C is a sectional view diagram of the filter 300 of FIG. 3 showing a relationship between the reducing agent 104 and the oxidizing agent 204 whereby electric field junctions 302 are formed, according to another embodiment 406.

Referring to FIGS. 1-4C, according to embodiments, the reducing agent 104 may be formed in a first continuous pattern 108. The oxidizing agent 204 may be formed in a second continuous pattern 208, and the first and the second continuous patterns 108, 208 may be disposed to have a plurality of crossing points, each crossing point forming an electric field junction 302 wherein a low voltage field is produced. In one embodiment, each crossing point forms an electric field junction wherein a low current flow is produced. In another embodiment, the first continuous pattern 108 of the reducing agent 104 includes a plurality of parallel lines in a first direction, and the second continuous pattern 208 of the oxidizing agent 204 includes a plurality of parallel lines in a second direction different than the first direction. The first continuous pattern 108 of the reducing agent 104 may include a grid including crossing lines in at least two axes, the second continuous pattern 208 of the oxidizing agent 204 may include a grid including crossing lines in at least two axes, and the first and the second continuous patterns 108, 208 may be rotated relative to one another to produce a plurality of crossing points, each crossing point forming an electric field junction 302. The use of grid patterns 108, 208 may be useful for maintaining electrical continuity throughout each of the reducing agent 104 and the oxidizing agent 108. For example, if any one segment of a given grid is broken, current passing through the pattern 108, 208 is shunted around the broken segment to maintain electrical continuity between opposite sides of the break. In one embodiment, at least one of the pattern 108 of the reducing agent 104 and the pattern 208 of the oxidizing agent 204 includes a metal conductor disposed to maintain electrical continuity of the pattern 108, 208.

Referring to FIG. 4A, in an embodiment, the first substrate 102 and the second substrate 202 are separate substrates, and the second surface 206 includes a surface of the second substrate 202 disposed against a back surface 408 of the first substrate 102.

Referring to FIG. 4B, in an embodiment, the first substrate 102 and the second substrate 202 are the same substrate, and the second surface 206 includes a side opposite from the first surface 106.

Referring to FIG. 4C, in an embodiment, the first substrate 102 and the second substrate 202 are separate substrates, and the second surface 206 includes a surface of the second substrate 202 disposed away from a back surface 408 of the first substrate 102.

According to another embodiment, referring to FIGS. 4A-4B, the system for filtering and treating air containing an aerosol or droplets further includes a third substrate 402 separate from the first substrate 202 and the second substrate 202.

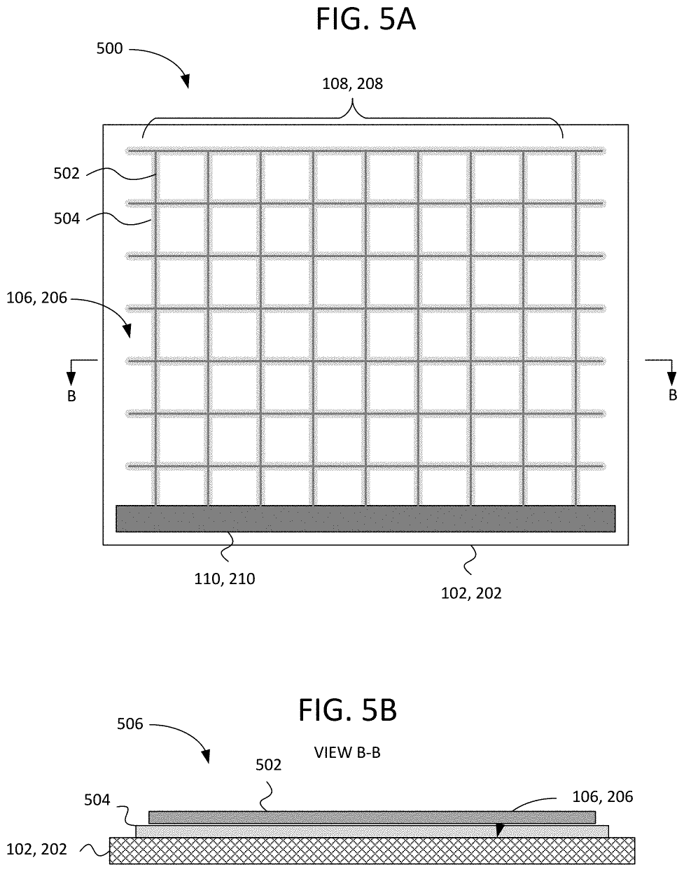

FIG. 5A is a plan view of a portion 500 of the filter 300 of FIG. 3 including a porous substrate 102, 202 showing a relationship between a conductor 502, which may be the reducing agent 104 of FIG. 1 or the oxidizing agent 204 of FIG. 2, and a dielectric 504 on a surface 106, 206 of the porous substrate 102, 202, according to an embodiment. FIG. 5B is a sectional view 506 of the portion 500 of the filter 300 of FIG. 3 including a porous substrate 102, 202 showing a relationship between a conductor 502, which may be the reducing agent 104 of FIG. 1 or the oxidizing agent 204 of FIG. 2, and a dielectric 504 on a surface 106, 206 of the porous substrate 102, 202, according to an embodiment.

According to an embodiment, referring to FIGS. 5A-5B, the system for filtering and treating air containing an aerosol or droplets further includes a dielectric material 502 layer disposed on the first and/or the second substrate 102, 202, the dielectric material 502 being disposed in a pattern between the reducing agent 104 and the oxidizing agent 204. In one embodiment, the dielectric material 502 comprises a substantially non-porous dielectric material. In another embodiment, the dielectric material 502 layer is disposed to cause the low voltage electric field junction 302 to form across a surface of the dielectric material 502 layer and through the first and/or the second porous substrates 102, 202. In another embodiment, the dielectric material 502 layer is formed in a pattern to raise an electrical resistance between the reducing agent 104 and the oxidizing agent 204.

Referring to all the figures above, in an embodiment, the second porous substrate 202 includes a paper material. In another embodiment, the second porous substrate 202 includes a non-woven fabric. Additionally and/or alternatively, the second porous substrate 202 includes a woven fabric. The second porous substrate 202 may include one or more of a polyester chiffon, a cotton material, or a polyester-cotton blend.

According to an embodiment, the first and the second substrates 102, 202 each include the same porous substrate. In an embodiment, each of the first and the second substrates 102, 202 are formed from a polyester chiffon material. Two layers of polyester chiffon plus one or more layers of cotton fabric (e.g., “quilter's cotton”) have been found to filter aerosols with substantially the same efficiency as an N95 mask, across all particle sizes. It is believed that two layers of polyester chiffon provide electrostatic attraction of aerosols nominally smaller than the open portion of the weave, to cause passing aerosols to adhere to the polyester chiffon.

According to embodiments, application of the low voltage electric field formed between the reducing agent 104 and the oxidizing agent 204 to the adhered aerosol causes virus particles carried by the aerosol to “denature” or otherwise be rendered non-infectious.

It is believed that the activity of the electric field on trapped virus particles may reduce the burden of training necessary to safely or adjust remove masks and respirators after use in an environment where virus may be present. If substantially all virus particles are converted to “dead” virus, then the amount of care needed to avoid contact with the filtering surface may be reduced.

According to an embodiment, the system for filtering and treating air containing an aerosol or droplets further includes a first current collector 110 in electrical continuity with the reducing agent 104, and a second current collector 210 in electrical continuity with the oxidizing agent 204. In one embodiment, the first current collector 110 is formed from the same material as the reducing agent 104, and the second current collector 210 is formed from the same material as the oxidizing agent 204.

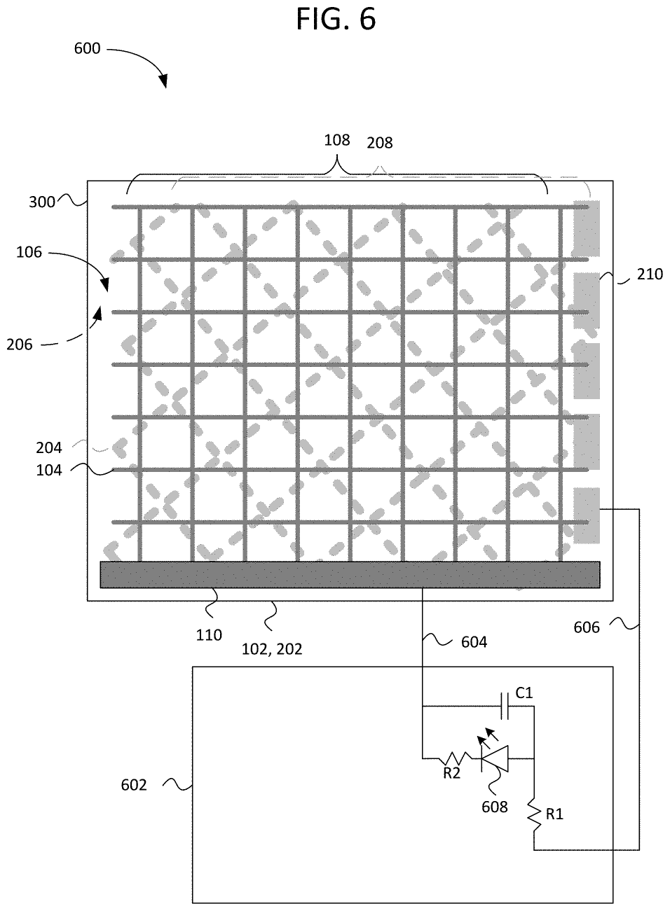

FIG. 6 is a diagram of a filter system 600 including a filter 300 operatively coupled to an electric circuit 602, according to an embodiment.

According an embodiment, referring to FIG. 6, the system 600 for filtering and treating air containing an aerosol or droplets further includes an electrical circuit 602 operatively coupled, respectively, to the reducing agent 104 and the oxidizing agent 204 via electrical leads 604 and 606. The electrical circuit 602 may include a voltage monitor circuit configured to energize an indicator 608 when an electrical field comprising the collective electric field junctions 302 is maintained by the filter 300.

In normal operation in a sufficiently humid environment, the zinc oxidizes by a low current flow of electrons from the zinc to the silver (such that current flows from the silver to the zinc). When measured across a high impedance circuit, the silver has a positive polarity and the zinc has a negative polarity when the low current flow is present. The voltage that forms between the silver and the zinc creates the low voltage electric field junctions 302 that cooperate to produce an overall low voltage electric field effective for rendering virus particles non-infective. When the zinc pattern and the silver pattern are continuous and respectively coupled to the voltage collectors 110, 210, the cumulative current flow may be used to power an LED 608 to indicate that the electric field junctions are active and capable of “killing” a virus. A simple circuit 602 that provides an indication of (passive) operation is shown in FIG. 6. The circuit 602 connects to the current collectors 110, 210, respectively in continuity with the zinc and the silver patterns, via the electrical leads 604 and 606. As the silver reduces, the resultant positive voltage on the electrical lead 606 may be harnessed to power a polarity-sensitive indicator 608 such as an LED. Operation of the LED 608 may be trimmed by an RC circuit including a resistor R2 and a capacitor C1, which operate as a simple timing circuit configured to cause the LED 608 to intermittently discharge, which provides a visible indication that the filter 300 is active and capable of rendering a filtered virus non-infectious. An optional offset resistor R3 may be selected to cause the silver to maintain a higher voltage and reduce the rate of consumption of the silver oxide.

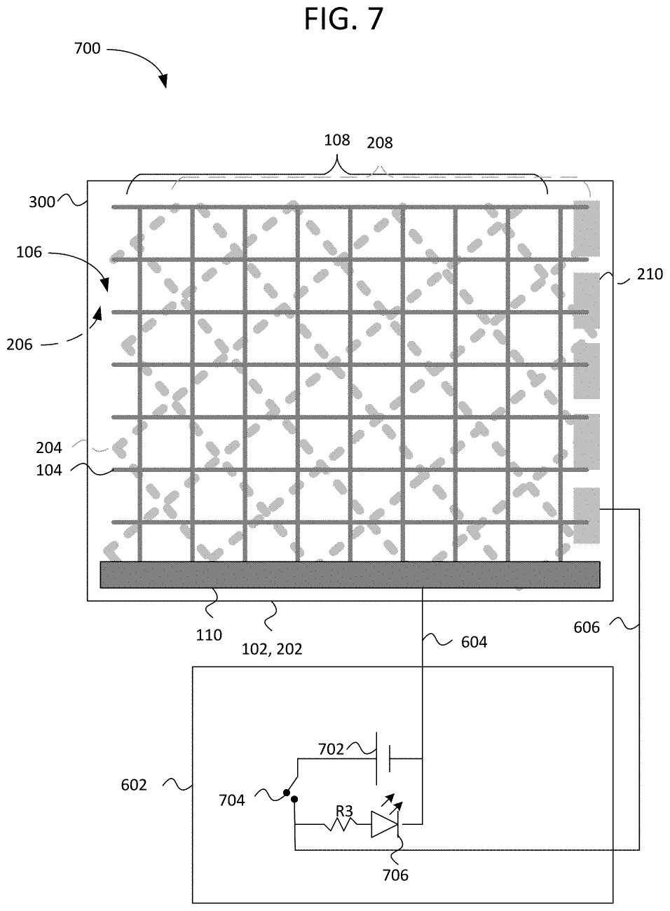

FIG. 7 is a diagram of a filter system 700 including a filter 300 operatively coupled to an electric circuit 602, according to an embodiment.

According to an embodiment, referring to FIG. 7, the electrical circuit 602 includes a charging circuit configured to cause oxidation of the oxidizing agent 204. In one embodiment, the charging circuit includes a battery 702 selectively coupleable by a switch 704 to apply a positive voltage to the oxidizing agent 204 and apply a negative voltage or ground to the reducing agent 104. In another embodiment, the charging circuit includes an indicator 706 configured to indicate when a charging voltage is being applied to the oxidizing agent 204.

According to an embodiment, the silver oxide and the zinc may be regenerated (e.g., “recharged”) to extend the time for the anti-virus effect of the silver and zinc electric field junctions 302. To re-charge or pre-charge the electric field junctions 302, the silver may be oxidized by application of a positive voltage from a battery 702, the applied positive voltage being selected to cause electrons to flow from the silver to the zinc (current to flow from the zinc to the silver). By selecting the proper voltage (e.g., about 3 volts), the electric field junction 302 field strength may be maintained, albeit reversed, so as to cause the filter 300 to maintain its anti-viral properties during regeneration.

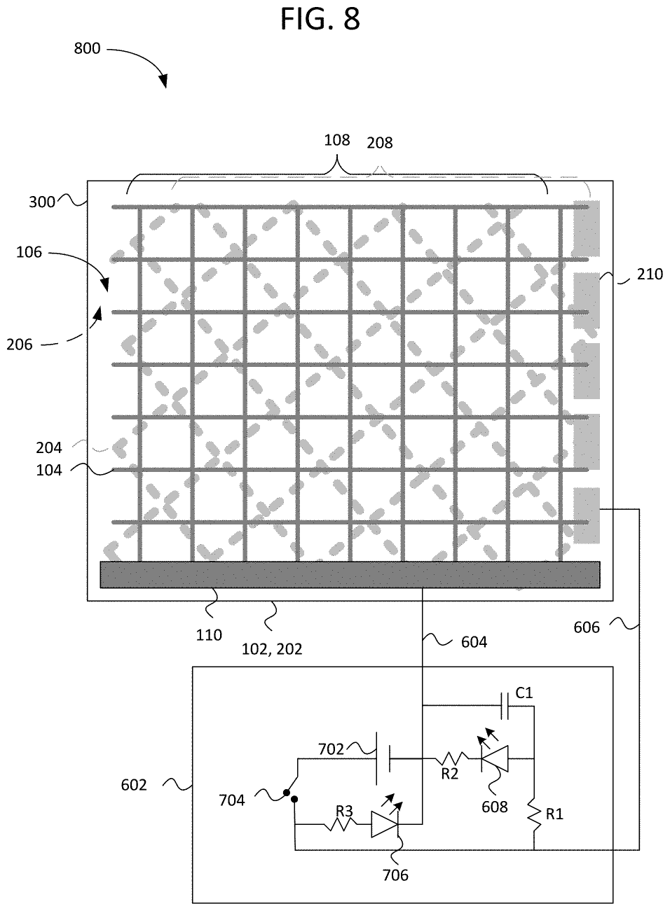

FIG. 8 is a diagram of a filter system 800 including a filter 300 operatively coupled to an electric circuit 602, according to an embodiment.

According to an embodiment, referring to FIG. 8, the electrical circuit 602 includes a voltage monitor circuit and a charging circuit.

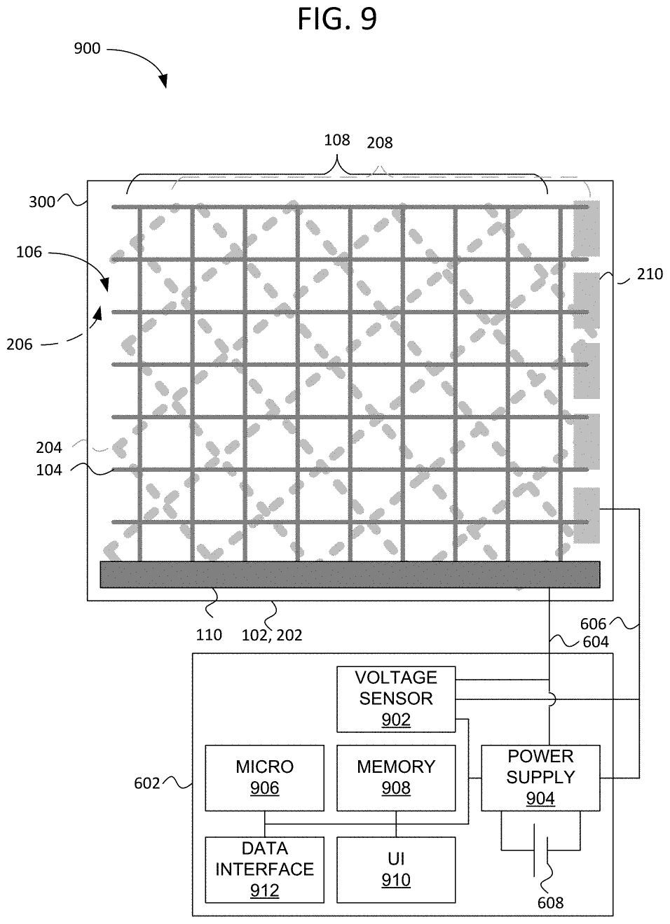

FIG. 9 is a diagram of a filter system 900 including a filter 300 operatively coupled to an electric circuit 602, according to an embodiment.

According to an embodiment, referring to FIG. 9, the electrical circuit 602 includes a digital circuit. The digital circuit may include a voltage sensor 902 configured to detect collective operation of the electric field junctions 302 between the reducing agent 104 and the oxidizing agent 204, a power supply 904 configured to apply a charging voltage, and a microprocessor or microcontroller 906 configured to control operation of the power supply 904 responsive to voltage detected by the voltage sensor 902. In one embodiment, the digital circuit includes memory 908 operatively coupled to the microprocessor or microcontroller 906. In another embodiment, the digital circuit includes a user interface 910 including one or more of a switch to enable operation of the digital circuit, a visible indicator to indicate operation of the filter and/or non-operation of the filter, and a haptic transducer to alert a user of the filter 300 of an operating condition. The digital circuit may include a data interface 912 configured to communicate with an external device. For example, the data interface 912 may include a Bluetooth transceiver operatively coupled to a personal electronic device such as a cell phone (not shown). In an embodiment, a user may control operation of the filter system 900 using an application installed on the personal electronic device. In an embodiment, a user may receive notice of a malfunction or an end-of-life condition of the filter system 900 via an application installed on the personal electronic device.

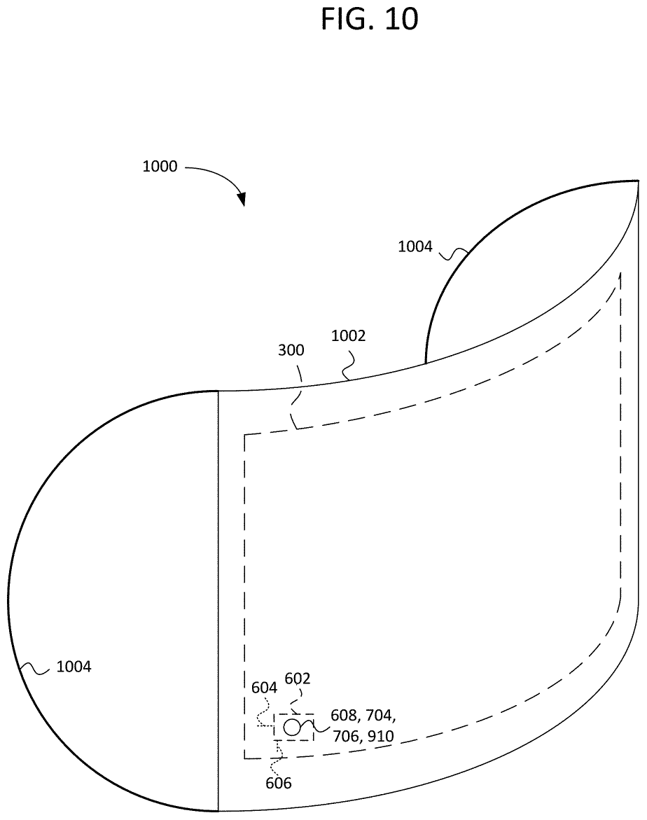

FIG. 10 is a diagram of a face mask 1000 including a covering 1002 and a filter 300 configured to filter aerosols and droplets carrying virus particles and to render the virus particles non-infectious, according to an embodiment.

According to an embodiment, referring to FIG. 10, the system for filtering and treating air containing an aerosol or droplets further includes a face mask 1000 including a filter 300 and an outer covering 1002 configured to be held over a user's nose and mouth with straps 1004. In one embodiment, the face mask 1000 further includes an electrical circuit 602 operatively coupled to the filter 300 via electrical leads 604, 606, the electrical circuit including at least one user interface object 608, 704, 706, 910.

Referring to FIGS. 1-10, a filter for inhibiting the spread of infectious disease includes at least one porous substrate, a first pattern carrying silver formed on the at least one porous substrate, and a second pattern carrying zinc formed on the at least one porous substrate. A first electrical lead is in electrical continuity with at least a portion of the first pattern. A second electrical lead is in electrical continuity with at least a portion of the second pattern. An electrical circuit is operatively coupled to the first and second electrical leads. In an embodiment, the silver and zinc are operatively coupled to form a reduction-oxidation reaction and at least one of electrical voltage or electrical current corresponding to the reduction-oxidation reaction between the first and second patterns is sufficient to render an infectious agent held by the porous substrate non-infectious.

The electrical circuit may be operable to draw current from the reduction-oxidation reaction through the first and second electrical leads. The electrical circuit may include a voltage detector and an indicator, such that the electrical circuit is configured to operate the indicator responsive to a detected voltage between the first and second electrical leads. The indicator may operate to alert the user that the zinc and silver are capable of a sufficiently strong reduction-oxidation reaction to render the infectious agent non-infections. In another embodiment, the indicator may operate to alert the user that the zinc and silver are respectively reduced and oxidized to a level where the reduction-oxidation reaction is reduced in strength.

The electrical circuit may includes a battery separate from the battery formed by the first and second patterns. In an embodiment, the electrical circuit is configured to apply current from the battery to the electrical leads responsive to a reduction in electrical potential between the first and second electrical leads. This may be used, for example to strengthen a reduced potential between the silver and zinc and/or to reverse the oxidation-reduction reaction to recharge the patterns.

In an embodiment, ambient or periodic moisture passed through the porous substrate enhances current flow between the silver and the zinc. In an embodiment, the filter for inhibiting the spread of infectious disease may include an electrolyte held by the porous substrate.

In an embodiment, the first and second electrical leads include a connector for detaching the electrical circuit from the porous substrate and the first and second patterns. The system may further include a pocket operatively coupled to the porous substrate, the pocket being configured to hold the electrical circuit. Accordingly, the electrical circuit may be removed from the porous substrate and the first and second patterns when the porous substrate is washed.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

1. A system for filtering and treating air containing an aerosol or droplets, comprising:
a first substrate;
a reducing agent formed on a first surface of the first substrate;
a second porous substrate formed from a porous material; and
an oxidizing agent formed on a second surface of the second porous substrate separate from the first surface;
wherein the reducing agent and the oxidizing agent are disposed in relationship to one another to form a plurality of electric field junctions between the reducing agent and the oxidizing agent; and
wherein the plurality of electric field junctions collectively form a low voltage electric field across the second substrate.

2. (canceled)

3. The system for filtering and treating air containing an aerosol or droplets of claim 1, wherein at least the second porous substrate is configured to filter at least a portion of an entrained aerosol or droplet when air is passed therethrough; and
wherein the low voltage electric field formed by the plurality of electrical field junctions performs treatment on an infectious particle carried by the aerosol or droplet.

4. (canceled)

5. The system for filtering and treating air containing an aerosol or droplets of claim 1, wherein the reducing agent comprises zinc.

6. The system for filtering and treating air containing an aerosol or droplets of claim 1, wherein the first substrate comprises a wire screen;
wherein the reducing agent comprises a layer of zinc disposed on the first substrate; and
wherein the first surface comprises substantially the entire surface of the wire screen.

7. The system for filtering and treating air containing an aerosol or droplets of claim 1, wherein the first substrate comprises a porous substrate; and
wherein the first surface of the first substrate comprises a first side of the first substrate.

8-10. (canceled)

11. The system for filtering and treating air containing an aerosol or droplets of claim 7, wherein the reducing agent is disposed in a continuous pattern.

12. (canceled)

13. The system for filtering and treating air containing an aerosol or droplets of claim 1, wherein the oxidizing agent comprises silver, Ag.

14. The system for filtering and treating air containing an aerosol or droplets of claim 13, wherein the oxidizing agent comprises silver, Ag and silver oxide Ag₂O.

15-16. (canceled)

17. The system for filtering and treating air containing an aerosol or droplets of claim 1, wherein the reducing agent is formed in a first continuous pattern;
wherein the oxidizing agent is formed in a second continuous pattern; and
wherein the first and the second continuous patterns are disposed to have a plurality of crossing points, each crossing point forming an electric field junction wherein a low voltage field is produced.

18. The system for filtering and treating air containing an aerosol or droplets of claim 17, wherein each crossing point forms an electric field junction wherein a low current flow is produced.

19-21. (canceled)

22. The system for filtering and treating air containing an aerosol or droplets of claim 1, wherein the first substrate and the second substrate are separate substrates; and
wherein the second surface comprises a surface of the second substrate disposed against a back surface of the first substrate.

23. The system for filtering and treating air containing an aerosol or droplets of claim 1, wherein the first substrate and the second substrate are the same substrate; and
wherein the second surface comprises a side opposite from the first surface such that the electric field junctions are formed through the first substrate.

24. The system for filtering and treating air containing an aerosol or droplets of claim 1, wherein the first substrate and the second substrate are separate substrates; and
wherein the second surface comprises a surface of the second substrate disposed away from a back surface of the first substrate such that the electric field junctions are formed through the first substrate and the second substrate.

25-31. (canceled)

32. The system for filtering and treating air containing an aerosol or droplets of claim 1, wherein the second porous substrate comprises a woven fabric.

33. The system for filtering and treating air containing an aerosol or droplets of claim 32, wherein the second porous substrate comprises a polyester chiffon.

34-36. (canceled)

37. The system for filtering and treating air containing an aerosol or droplets of claim 1, further comprising:
a first current collector in electrical continuity with the reducing agent; and
a second current collector in electrical continuity with the oxidizing agent.

38. The system for filtering and treating air containing an aerosol or droplets of claim 37, wherein the first current collector is formed from the same material as the reducing agent; and
wherein the second current collector is formed from the same material as the oxidizing agent.

39. The system for filtering and treating air containing an aerosol or droplets of claim 1, further comprising:
an electrical circuit operatively coupled, respectively, to the reducing agent and the oxidizing agent via electrical leads.

40. The system for filtering and treating air containing an aerosol or droplets of claim 39, wherein the electrical circuit includes a voltage monitor circuit configured to energize an indicator when an electrical field comprising the collective electric field junctions is maintained by the filter.

41. The system for filtering and treating air containing an aerosol or droplets of claim 39, wherein the electrical circuit comprises a charging circuit configured to cause oxidation of the oxidizing agent.

42-43. (canceled)

44. The system for filtering and treating air containing an aerosol or droplets of claim 39, wherein the electrical circuit includes a voltage monitor circuit and a charging circuit.

45-48. (canceled)

49. The system for filtering and treating air containing an aerosol or droplets of claim 1, further comprising a face mask including a filter and an outer covering configured to be held over a user's nose and mouth with straps.

50. The system for filtering and treating air containing an aerosol or droplets of claim 49, wherein the face mask further comprises:
an electrical circuit operatively coupled to the filter via electrical leads, the electrical circuit including at least one user interface object.

51-57. (canceled)
